(19) United States
(12) Patent Application Publication
(10) Pub. No.: US 2006/0049932 A1
(43) Pub. Date: Mar. 9, 2006

(54) Bio-chemical beeper

(76) Inventors: Diahann Grasty, Phoenixville, PA (US); Laniya Rose Clark Grasty, Phoenixville, PA (US)

Correspondence Address: Diahann Grasty, 411 Bill Smith Blvd., King of Prussia, PA, 19406, US

(21) Appl. No.: 10/735,296

(22) Filed: Aug. 17, 2004

(51) Int. Cl. G08B 19/00; A61B 5/00; G08B 1/00; G08B 23/00; G08C 19/16; G06F 19/00

(52) U.S. Cl. 340/521; 340/573.1; 340/531; 340/870.01; 600/300; 702/19; 702/22

(57) ABSTRACT

This Bio-Chemical Beeper will be used to locate, detect, distinguish any chemical, gas or radiation or any foreign substance. The system will by color/data/alpha/numeric/video/midi/audio/satellite detect and relay the results to the appropriate security system, paramedics, and will send a message to a home base picture frame used for entertainment, allowing cellular capabilities as well. This system will be internet and satellite capabilities, voice activation as well as dial up and keypunch This system will send and receive information data/alpha/numeric/video audio/midi/satellite with the capability to print. The locator system will use satellite to find this system and keep track of the secondary system used to locate an individual, show the users name and address, blood type, allergies and any health conditions. In the case of an extreme disaster the loss of connection will signal a separate database of loss units.

### STATEMENT REGARDING FEDERALLY SPONSORED RESEARCH OR DEVELOPMENT

DCED grants non-exclusive licenses on approximately 1,500 doe owned U.S. Patent and DOE owned foreign Patents to U.S. Citizens and Corporations. Individual firms, or corporation with satisfactory plans for development, and or marketing of the invention.

### CROSS-REFERENCE TO RELATED APPLICATION

| | | | | |
|---|---|---|---|---|
| 6233698 | 6232780 | 6225989 | 6219573 | 6216001 |
| 6215403 | 6200443 | 6198695 | 6178338 | 6172759 |
| 6354996 | 6353413 | 6348694 | 6346886 | 6345260 |
| 6340117 | 6335679 | 6332085 | 6328212 | RE37467 |
| 6327477 | 6327343 | 6321989 | 6301366 | 6288646 |
| 6287510 | 6277071 | 6255951 | 342/357.07 | 340/825.72 |
| 358/513 | 358/514 | 356/399 | 250/338.5 | 250/339.08 |

### REFERENCE TO A MICROFICHE APPENDIS

N/A

### BACKGROUND OF THE INVENTION

This invention relates to a Bio-chemical beeper/cell phone for relaying data, alphanumeric/audio/visual to all security systems, hospital and paramedics, including the proper authorities to react to the detection sent by the Bio-chemical beeper/cell phone and any chemical or dangerous substance. Radiation, sound waves and any type of energy that will due harm.

Bio-chemical beeper/cell phone have not been addressed as of yet and the demand for this intention is extremely in demand with the threat of chemical warfare and terrorism, child abduction, hijackers and etc. This Bio-chemical beeper/cell phone will assist in the location, detection and alpha/audio/video/visual description and remedies to protect as many lives as possible.

This Bio-chemical beeper/cell phone's objection is to provide emergency contact to be sent out alpha/audio/video/numeric to the proper authorities, in case of bio-chemical warfare, detecting the chemical sending out information of the location and the type of the chemical it has come into contact with, with audio/video/data of the location to be sent too, and will allow audio/video/data to be received by the proper authority. It will have direct contact with paramedics and security systems and 911 services to provide as much information as possible. There will be printable capabilities and alpha/audio/video capabilities.

Once it comes in contact with gas, liquid, fumes and powder or any life threatening substance or radiation. This system will distinguish the components of the chemical and send that signal to the closest security system for that area with the location, the type of chemical and or substance or radiation. Based on the chemical components the beeper has detected the signal for the locator will be activated and the system will dial into the nearest security system giving the location and the type of chemical it has detected. This will be done by internet capabilities or satellite for location and chemical components and information. When the system identifies the chemical through color coding or what ever means that is necessary and connects to a database or the appropriate apparatus it will relay the information back to the original beeper to allow people to have the necessary information to save and locate as many people as possible until help arrives. The message will be continuous and will also allow the user of the beeper to respond to the message that is being sent with touch screen capabilities as well as keypunch/voice activation.

The security system will notify the appropriate authorities and give them the beeper number that will allow them to interface with the beeper including paramedics to give access to the beeper and give assistance while on route to the disaster area and also, identify the user. This system can also be used to locate kidnapped and abducted children with a special separate device that interfaces with the bio-chemical beeper/cell phone that will not be recognizable such as a button, hair clip or pin including a shoelace and or buckle, snap etc. once the main system has been activated. All children locator will be different for security purposes for tracking similar to a tracking device.

The bio-chemical beeper/cell phone is comprised of the necessary sensors and material that will be necessary to detect the chemical and or substance, radiation etc. Transmit the type of chemical it will also include a battery indicator, off and on switch, send signal and a receive signal and print command signal and a digital/video cameras that will send video communication, internet and satellite and print command signals using the on screen touch capability as well as keypunch and voice activation and can be used as a cell phone.

The bio-chemical beeper will be programmable for any new bio-chemical substance that comes into the market, by color or any other code necessary. The beeper/cell phone will be virus protected, as well as, allowing the system to read the components of the bio-chemical beeper will also be used for the home as a picture frame to store video that are sent as well as a detection system.

We claim:

1. The interface between the substance, radiation or any foreign chemical to the human body, that is detected and color/data/alpha or numeric/video/midi/audio coded. Using a transmitter/receiver/satellite any conducer or condenser or chips or what ever is necessary to make this feasible.

2. We claim the interface of the color/data/alpha or numeric/midi/audio/video coded system that will relay to the appropriate database to contact the security system, Internet.

3. We claim the update of the programmable database to detect and distinguish the foreign substance.

4. We claim all databases necessary to transmit/dial out to the appropriate security system, paramedics and to receive information back into the system.

5. We claim the use of any chips, infrared lights, satellite and transmitters, receivers used to make this beeper/cell phone feasible.

6. We claim all relay messaging through satellite, towers, electrical, midi, video, audio, wave including internet and cellular messaging.
